(19) United States
(12) Patent Application Publication
(10) Pub. No.: US 2015/0066077 A1
(43) Pub. Date: Mar. 5, 2015

(54) Device For Occluding An Opening In A Body And Associated Methods

(71) Applicant: Occlutech Holding AG, Schaffhausen (CH)

(72) Inventor: Mehmet Hakan Akpinar, Instanbul (TR)

(21) Appl. No.: 14/381,923

(22) PCT Filed: Feb. 28, 2013
(86) PCT No.: PCT/EP2013/054112

§ 371 Date: Aug. 28, 2014

Related U.S. Application Data
(60) Provisional application No. 61/605,102, filed on Feb. 29, 2012.
(60) Provisional application No. 61/706,698, filed on Sep. 27, 2012.

(51) Int. Cl. A61B 17/00

(52) U.S. Cl. CPC A61B 17/0057; A61B 2017/00243; A61B 2017/00575; A61B 2017/00592; A61B 2017/00597; A61B 2017/00606
USPC 606/213

(57) ABSTRACT

The disclosure relates to a device for occluding an opening in a body and associated methods. The device of this disclosure has the advantage that a second element of the device can be made more flexible than a first element and thereby the device is less prone to increase the rupture size of an opening. In one example, a device (1) for occluding an opening in a body is disclosed, which comprises a first element (2), comprising at least one thread or wire (3), and a second element (6), such as a shaft section, said second element (6) being at least in a longitudinal section tubular or oval and comprising a braided material, said braided material comprising at least one thread or wire (9), and wherein a diameter of said second element (6) is of substantially the same size or smaller than said opening for all longitudinal sections, when said device (1) is in a preset, expanded configuration.

### FIELD OF THE INVENTION

This disclosure pertains in general to the field of medical implants or endoprostheses. More particularly the disclosure relates to a device for occluding an opening in a body and associated methods.

### BACKGROUND OF THE INVENTION

The present disclosure is related to the sealing of an opening in a body vessel or the wall of a heart cavity, e.g. a blood vessel or a human heart, and more precisely to a device for occluding an opening in a body, and a method for manufacturing such a device.

There are holes and openings not created by interventional treatment activity, acquired as a result of disease or congenital. Some products for closing acquired or congenital defects are devices having umbrella shaped discs with spikes and a covering cloth. One disc is placed on one side of the opening to be closed and another disc is placed on the other side of the opening to be closed. Thereafter, the discs are pressed against each other and locked to seal the opening. StarFlex® (NMT Medical Inc®, Boston Mass.) and CARDIA Patent Foramen Ovale Closure Device® (Cardia Inc®, Burnsville, Minn.) are such devices.

Other devices are made of Nitinol threads and have a double disc shape with a waist between the discs. They are inserted in openings that are to be closed, one disc on each side of the hole that are to be closed and the waist in the center of the hole, the discs being larger than the hole. There are two examples of such devices. The first, made by Occlutech® having one fixation point at the end of the device and the second, made by AGA medical® having two fixation points, one at each end of the device. In these devices, the Nitinol threads are joined in the centre of one or both of the discs. These devices work well when the hole or opening is circular and the wall of the opening is thin.

However, some defects, e.g. ventricular septal defects, are not circular holes or openings. Instead there may be ruptures, for instance with torn tissue. Ventricular walls are substantially thicker tissue structures than septal walls. Such ruptures in structures like these may for instance occur, after a myocardial infarct or as part of a muscular ventricular septal defect.

When using a prior art occluder for closing one of the passages of such complicated defects, it may be difficult to completely seal such a gap of an opening, thus the prior art devices do not always work well with these kind of ruptures, since they sometimes may further tear tissue that has already been torn, and thus may increase the rupture size.

Moreover, when it comes to ruptures, the prior art devices may not always cover all of the shunts or channels.

Furthermore, the prior art devices may for some defects, such as ruptures, cover an unnecessary large portion of healthy tissue. This may sometimes be undesired from a necrosis point of view.

Thus, there is a need of an improved device and/or associated method or procedure, which works well with ruptures and does not increase the rupture size.

There is also a need of an improved device and/or associated method or procedure, which can completely fill the gap of a rupture to eliminate residual shunts.

Furthermore, there may also be a need of an improved device and/or associated method or procedure, which covers healthy tissue to a less extent than prior art devices.

### SUMMARY OF THE INVENTION

Accordingly, examples of the present disclosure preferably seek to mitigate, alleviate or eliminate one or more deficiencies, disadvantages or issues in the art, such as the above-identified, singly or in any combination by providing a device for occluding a body opening, a method of manufacturing a device for occluding an opening in a body and a medical procedure for occluding an opening in a body, according to the appended patent claims.

A disadvantage with the prior known devices for occluding an opening in a body is that when the device has a single body, the middle section, which can be called the waist, is circular and this middle section gets a lot of support from the right and left disc-shaped end sections in order to keep its shape circular, while trying to keep its shape memory. The reason that the middle section gets a lot of support from the end sections is that the different sections are all part of one integrated unit, produced the same way and comprising the same material, with the same size. Thereby the design is not flexible enough. The present disclosure overcomes this and/or other disadvantages with prior art by providing a device for occluding an opening in a body, in which device a second element of the device can be made more flexible than a first element and thus provide for an elastic design suitable for both systolic and diastolic thickness of the ventricular septum.

According to one aspect of the disclosure, a device for occluding an opening in a body is provided. The device comprises a first element, comprising at least one thread or wire. The device may comprises and a second element, such as a shaft section. The second element may be tubular. It may be at least in a longitudinal section circular. Alternatively, it may be oval at least in a longitudinal section. The second element comprises a braided material of helically wound strands in a longitudinal direction. The braided material comprises at least one thread or wire. A diameter of the second element is of substantially the same size or smaller than the opening to be occluded for all longitudinal sections, when the device is in a preset, expanded configuration.

According to another aspect of the disclosure, a method of manufacturing a device for occluding an opening in a body is provided, which method comprises forming a first section of a first element or part thereof; forming a second section of a first element, or part thereof; forming a third section of a first element or part thereof; and optionally joining different parts. The method further comprises forming first, second and third sections of a second element by braiding so that a diameter of the second element is of substantially the same size or smaller than the opening for all longitudinal sections, when the device is in a preset, expanded configuration; and joining the first and second elements.

According to yet another aspect of the disclosure, a medical procedure for occluding an opening in a body is provided, which medical procedure comprises positioning a device inside a restraining catheter; and positioning a pushing catheter inside the restraining catheter adjacent to the device, further away from a target site than the device. Then, the restraining catheter, the pushing catheter and the device are inserted into the body. A distal end of the restraining catheter is positioned at the target site and the device is positioned inside the bodily opening. The device is pushed through the restraining catheter with the pushing catheter until the device has been released, so that a first section of a first element of the device is positioned on an inside of a rupture to be sealed. The pushing catheter is removed. The restraining catheter is removed, so that the first section of the first element of the device is positioned on the inside of the rupture to be sealed with the device and a third section of the first element is positioned on an outside of the rupture. A second element of the device is returned to its preset shape and the device thereby is radially contracted so as to close the rupture. The restraining catheter is then removed from the body.

Further examples of the disclosure are defined in the dependent claims, wherein features for the second and subsequent aspects of the disclosure are as for the first aspect mutatis mutandis.

Some examples of the disclosure provide for a decreased material cost.

Some examples of the disclosure provide for less coverage of healthy tissue.

Some examples of the disclosure enable that the first and second elements can be manufactured in different ways independent of each other and thus with completely different properties, materials or size, such as diameter of thread or wire.

Some examples of the disclosure also provide for a second element, made of braided material, which can be made more flexible and thereby the device is made less prone to increase the rupture size of an opening, and the device can fill the gap completely to eliminate residual shunts or channels.

Some examples of the disclosure also provide for there being need for only very little radial strength in a second element of the device.

Some examples of the disclosure provide for a second element of the device, which has a lower expansive force than a first element.

Some examples of the disclosure provide for a second element of the device, which second element, and device, does neither tear a body opening nor further tear an already torn body opening.

Some examples of the disclosure provide for the assembling of a device from different, separately designed elements, so that the strength of the disc-shaped section of the first element does not affect the strength of the second element and the second element can stay oval with a very small retention force, caused by the shape memory.

Some examples of the disclosure provide for an elastic design, which works well with both sistolic and diastolic thickness of the ventricular septum.

Some examples of the disclosure enable both of the disc-shaped sections of a first element and a third element to be larger, i.e. have a larger diameter, and still be made up of less material, which is advantageous, since the material cost will be decreased and since healthy tissue will not be covered by the device to the same extent as prior art devices.

Some examples of the disclosure provide for allowing a sliding movement of the first element relative the second element.

Some examples of the disclosure also provide for enhancing the adhesion and proliferation of fibroblasts and endothelial cells onto surfaces.

Some examples of the disclosure also provide for overcoming certain materials cytotoxic issues.

Some examples of the disclosure also provide for faster endothelization.

Some examples of the disclosure also provide for a symmetrical design

Some examples of the disclosure also provide for a simplified manufacturing.

It should be emphasized that the term “comprises/comprising” when used in this specification is taken to specify the presence of stated features, integers, steps or components but does not preclude the presence or addition of one or more other features, integers, steps, components or groups thereof.

A ventricular septal defect is a defect in the ventricular septum, the wall dividing the left and right ventricles of the heart. The ventricular septum consists of an inferior muscular and superior membranous portion and is extensively innervated with conducting cardiomyocytes.

A muscular ventricular septal defect is a defect of the inferior muscular portion.

A postinfarction ventricular septal defect or myocardial infarction complications may occur immediately following a heart attack, e.g. a rupture of the intraventricular septum, the muscle separating the left and right ventricles, causing a ventricular septal defect with shunting of blood through the defect from the left side of the heart to the right side of the heart, which can lead to right ventricular failure as well as pulmonary overcirculation.

Perimembranous ventricular septal defects are located in the left ventricle outflow tract beneath the aortic valve.

Membranous ventricular septal defects are more common than muscular ventricular septal defects, and are the most common congenital cardiac anomaly.

Specific devices and methods described herein are applicable to treating at least one of the aforementioned defects, amongst others.

### BRIEF DESCRIPTION OF THE DRAWINGS

FIG. 1 is a top view in which an example of a device is illustrated for occluding an opening in a body.

FIG. 2 is a lateral view in which an example of the device is illustrated for occluding an opening in a body.

FIG. 3 is an elevated view illustrating an example of the device for occluding an opening in a body from the top at an angle.

FIG. 4 is another view in which the device is illustrated for occluding an opening in a body, seen from the top at an angle.

FIG. 5 is an elevated view in which an example of a part of a first element of a device for occluding an opening in a body from the top at an angle is illustrated.

FIG. 6 is an elevated view in which an example of a first element of a device for occluding an opening in a body is illustrated from the top at an angle.

FIG. 7 is another elevated view in which an example of a device for occluding an opening in a body is illustrated from the top at an angle.

FIG. 8 is yet another view in which an example of a device for occluding an opening in a body is illustrated from the top at an angle.

FIG. 9 is a flowchart in which steps of an exemplary method of manufacturing a device for occluding an opening in a body is illustrated.

FIG. 10 is a view in which an example of a device is illustrated, for occluding an opening in a body, in its at least partly collapsed state.

FIG. 11 is a schematic view in which examples of catheters for a medical procedure are illustrated.

FIG. 12 is a lateral view in which an example of a device for occluding an opening in a body is illustrated.

FIG. 13 is a top view in which a second element of an example of a device for occluding an opening in a body is illustrated.

FIG. 14 is another top view in which an example of a second element of a device for occluding an opening in a body is illustrated.

FIG. 15 is a lateral view in which an example of a device for occluding an opening in a body is illustrated.

### DESCRIPTION OF EXAMPLES

Specific examples of the disclosure will now be described with reference to the accompanying drawings. This disclosure may, however, be embodied in many different forms and should not be construed as limited to the examples set forth herein; rather, these examples are provided so that this disclosure will be thorough and complete, and will fully convey the scope of the disclosure to those skilled in the art. The terminology used in the detailed description of the examples illustrated in the accompanying drawings is not intended to be limiting of the disclosure. In the drawings, like numbers refer to like elements.

The following description focuses on an example of the present disclosure applicable to occlusion of a body opening and in particular to a device for occluding a body opening, such as a body opening associated with a postinfarctation ventricular septal defect. However, it will be appreciated that the disclosure is not limited to this application but may be applied to many other situations, including for example occlusion of a body opening associated with a perimembranous ventricular septal defect or a muscular ventricular septal defect.

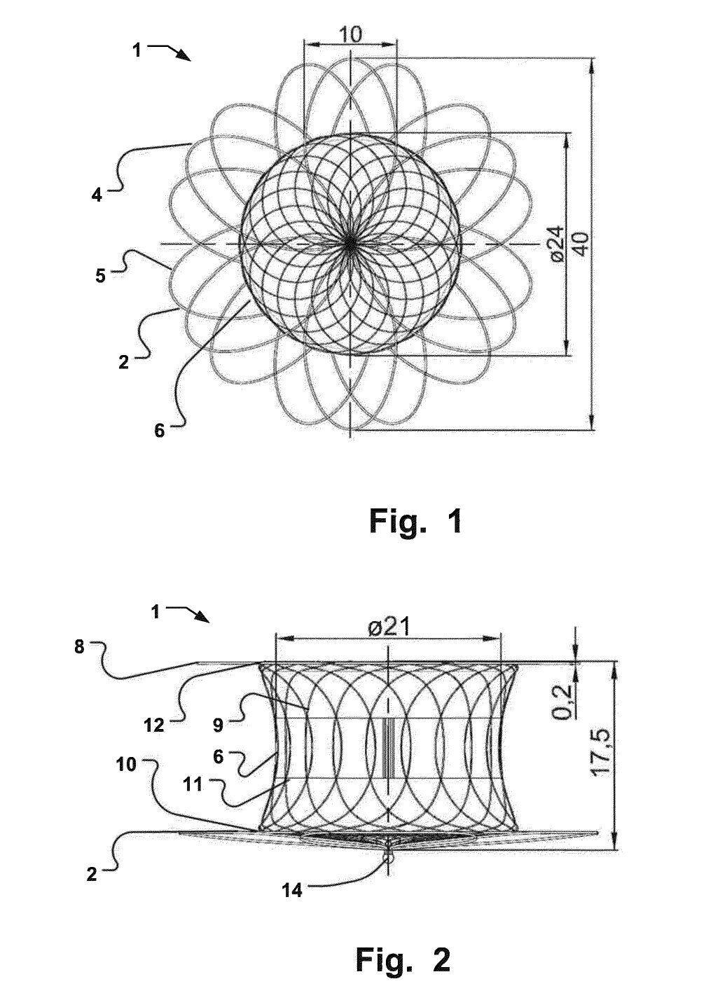

In an example of the disclosure according to FIG. 1, a device 1 is provided with a first element 2. The element 2 comprises at least one thread, strand or wire 3, i.e. the element can be made up of one sufficiently long thread or wire, but it can also be made up of a plurality of threads or wires, that have been put together to form the element 2. The element 2 also comprises a first section 4, which section 4 is normally not braided, but shaped like a disc, i.e. extending substantially in one geometrical plane. The first section 4 comprises at least one loop 5 of the thread or wire 3 extending from a central portion of the first section 4 to a peripheral portion of the first section 4 and returning back to the central portion.

By forming the first element this way, instead of forming it from braided material as it has been done in prior art, the first element can be larger and still be made up of less material than prior art, which is advantageous, since the material cost will be decreased and since healthy tissue will not be covered by the device to the same extent as prior art devices.

The exemplary device 1 illustrated in FIGS. 1 and 2 with sizing measures. The dimension of the sizing measures is [mm]. Relative dimensions of the example can thus be determined from the drawings if so desired. However, the absolute size measures are only to be seen as exemplary numbers of a specific example of a device 1.

The device 1 is also provided with a second element 6, such as a shaft section. The second element 6 shown in FIG. 1 is also shown from the side in FIG. 2. The second element 6 comprises a braided structure. The braided structure comprises and is made of at least one braided thread or wire 9, i.e. it can be made up of one sufficiently long thread or wire, but it can also be made up of a plurality of threads or wires. The second element 6 is at least in a longitudinal section 11 tubular and may be substantially circular or alternatively oval and comprises three different sections, one first section 10 at the end of the element, one second section 11, which is tubular and circular or may be oval, in the middle and one third section 12 at the other end of the second element 6. In some examples, a cross-sectional dimension, such as the diameter, of the second element 6 and/or the second section 11 thereof is of the same size, substantially the same size or smaller than the opening to be occluded by the device 1 for all longitudinal sections, i.e. first, second and third sections 10, 11, 12 of the second element 6, while the device is in a preset, expanded configuration. The longitudinal section 11 of the second element 6 can have a non-concentric oval or elliptic shape in a cross-section perpendicular to a centre axis of the longitudinal section. Due to this feature, a more flexible movement in a longitudinal direction is provided. Thus, the device 1 more easily moves with the movement of the surrounding tissue, i.e. with the movement according to the systole and the diastole. Thus, the device 1 adapts to the changes of the defect, since the device 1 move in a longitudinal direction at the same time as the cross-sectional size of the second element 6 is reduced. Therefore, the rupture is not increased, which may be the case if instead the longitudinal section has a round cross-sectional shape, since a rupture normally has a more elongated non-round shape.

By providing the device 1 with this second element 6, the device 1 or at least the second element 6, comprising braided material, can be made more flexible and thereby the device 1 will be less prone to increase the rupture size of an opening, when used to seal the opening. Furthermore, the device 1 can fill the gap of an opening completely to eliminate residual shunts or channels. Such residual shunts or channels may occur, when the opening is a rupture. Moreover, there is less radial strength in the second element 6 than in the first element 2, since the second element 6 is made of braided material. Another contributing reason for less radial strength in the second element 6 could be that the diameter of the thread or wire 9 can be made smaller than the diameter of the first element 2. Yet another contributing factor to the lower radial strength in the second element 6 could be due to the use of a different material, i.e. the material used for the first element 2 may be stiffer than a material used for the second element 6. Furthermore, by providing the device 1 with this second element 6, part of the device 1, i.e. the second element 6, can be made to have lower expansive force than the first element 2.

In some examples, the device 1 is also equipped with an adapter 14 for connecting the device 1 to a wire for delivery and/or retrieval. The first element 2 of the device can be formed substantially as a cone with the centre of the cone extending slightly inwardly towards the centre of the device. Thus, the adapter 14 is sunk in into the device 1 and will not impede blood flow in the vessel, where it is situated.

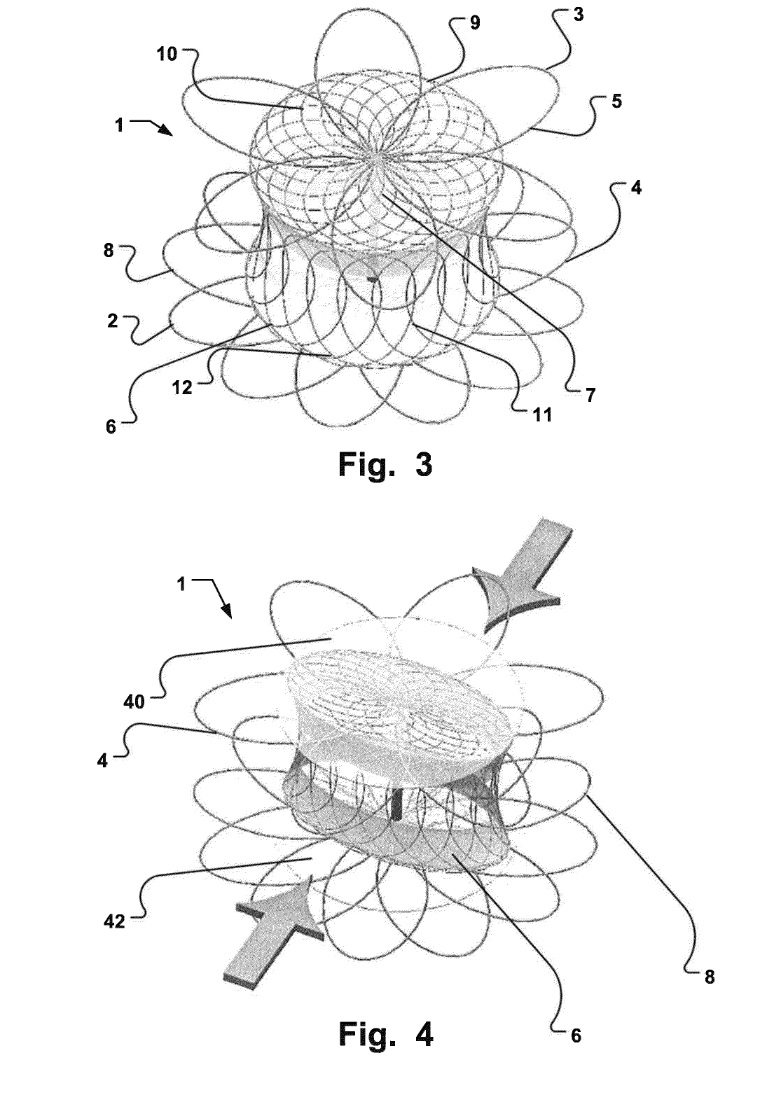

From FIG. 3 it can be seen that the braided material is braided so that the at least one thread or wire 9 of the braided material has a certain first braid pitch in a first section 10, i.e. an end section, of the second element 6. From FIG. 3, it can also be seen that in a second section 11, i.e. a middle section, of the second element 6, a certain second braid pitch, different from the first braid pitch, has been used. Thus, the second section 11, i.e. the middle section, of the second element 6 can be made more flexible than the first section of the second element 6, and thereby facilitating stretching of the second section 11. The second braid pitch can be larger or smaller than the first braid pitch. In order to make the second section 11 of the second element more flexible than the first section 10 of the second element, instead of using a different braid pitch, a lower number of threads or wires 9 can be used for the second section 11 of the second element than for the first section 10 of the second element.

The first element 2 can further comprise a second section 7, such as a tubular section, which is connected to the first section 4. The second section 7 of the first element 2 is shown in FIG. 6. The first section 4 has a larger diameter than a diameter of the second section 7.

The second element 6 does neither tear a body opening nor further tear an already torn body opening, since the second element 6 can be compressed in a different way than the first element 2 and since the second element 6, with braided material, can be compressed without deforming the first element 2.

By assembling a device from different, separately designed elements (first 2 and second 6), the strength of the disc-shaped section of the first element 2 does not affect the strength of the second element 6 and the second element 6 can stay oval with a very small retention force, which force can be supplied solely by the use of a material, which has shape memory properties.

Furthermore, by assembling a device from different elements (first 2 and second 6), an elastic design, which is suitable for both sistolic and diastolic thickness of the ventricular septum can be provided.

Optionally, the first element 2 may further comprise a third section 8, such as a non-braided disc-shaped section. The third section 8 of the first element is then connected to the second section 7 of the first element 2 and this third section 8 comprises at least one loop 5.

If the first element 2 of the device 1 comprises a third section 8, then both of the disc-shaped sections, i.e. the first section 2 and the third section 8, of the first element 2 can be larger, i.e. have a larger diameter and still be made up of less material, which is advantageous, since the material cost will be decreased and since healthy tissue will not be covered by the device 1 to the same extent as prior art devices.

Furthermore, in some examples, at least one loop 5 of the first section 4 is shaped as a petal and at least one loop 5 of the third section 8 is shaped as a petal. All of the loops 5 may be shaped as petals.

Moreover, in some examples at least one thread or wire 3 is used for the first, second and third sections 4, 7, 8 of the first element 2, so that the at least one thread or wire 3 runs through the first, second and third sections 4, 7, 8 of the first element 2.

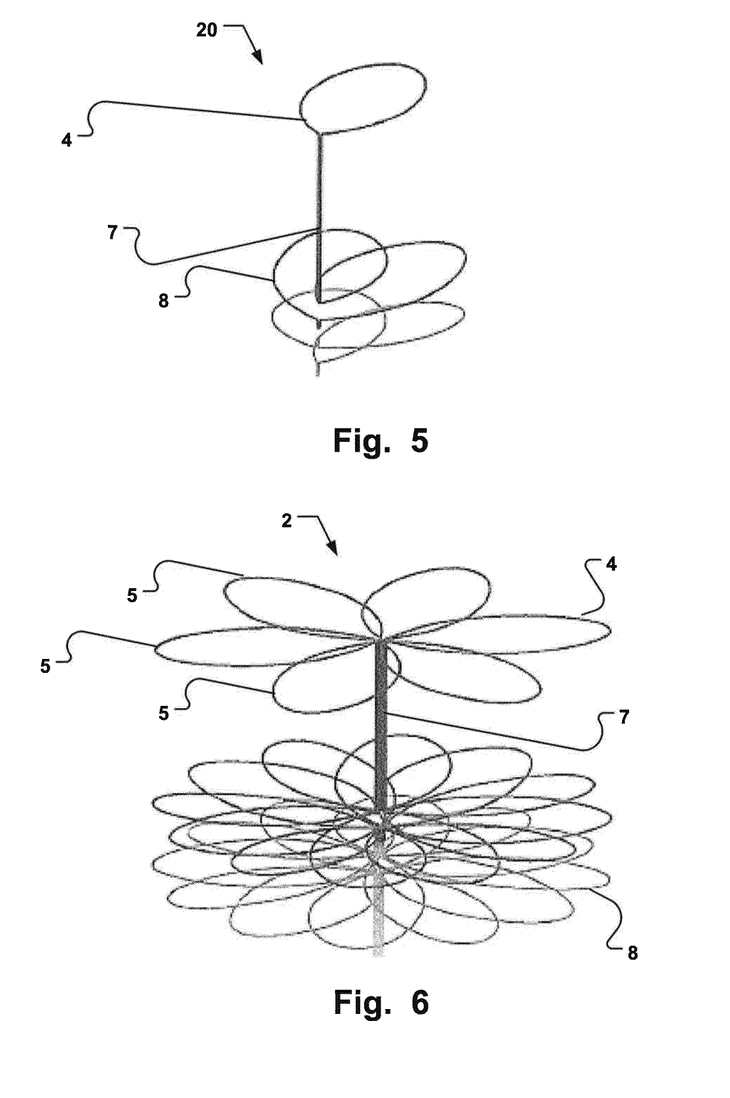

This is illustrated in FIGS. 5 and 6. FIG. 5 shows at the top, the first section 4 of the first element 2. The second section 7 and the third section 8 can also be seen from FIG. 5. The first section 4 is connected to the second section 7 and the second section 7 is connected to the third section 8. In FIG. 5, the first section 4 comprises only one loop or petal, whereas the third section comprises two overlapping loops or petals. However, any number of loops or petals can be used in the first and the third sections. In the example depicted in FIG. 5, a single, sufficiently long thread or wire is used for all of the different sections 4, 7, 8, i.e. only one thread or wire is used to produce the part, comprising the first, second and third sections 4, 7, 8 of the first element 2.

From FIG. 6, another example is shown, in which the first section 4 of the first element 2 can be seen at the top. The second section 7 and the third section 8 can also be seen in FIG. 6. The first section 4 is connected to the second section 7 and the second section 7 is connected to the third section 8. In FIG. 6, the first section 4 comprises a plurality of loops 5 or petals, whereas the third section 8 comprises another plurality of overlapping loops 5 or petals. Any number of loops 5 or petals can be used in the first and the third sections 4, 8 and the loops or petals may or may not be overlapping each other. In an example depicted in FIG. 6, a single, sufficiently long thread or wire can be used for all of the different sections 4, 7, 8, i.e. only one thread or wire is needed to produce the first element 2, comprising the first, second and third sections 4, 7, 8. However, in another example, the first element 2 can also be assembled from different parts, such as the part 20 shown in FIG. 5. As shown in FIG. 5, the part 20 may comprising parts of the first, second and third sections 4, 7, 8 of the first element 2. In this example, the plurality of loops 5 are formed from a plurality of threads or wires, and the plurality of threads or wires are joined by a technique, such as welding, pinching the plurality of threads or wires together, clamping the plurality of threads or wires together, or hooking the plurality of threads or wires together. By using a plurality of threads or wires in order to manufacture different parts, such as the part 20 in FIG. 5, and thereafter putting the different parts together, so that they will form a complete first element 2, manufacturing is simplified.

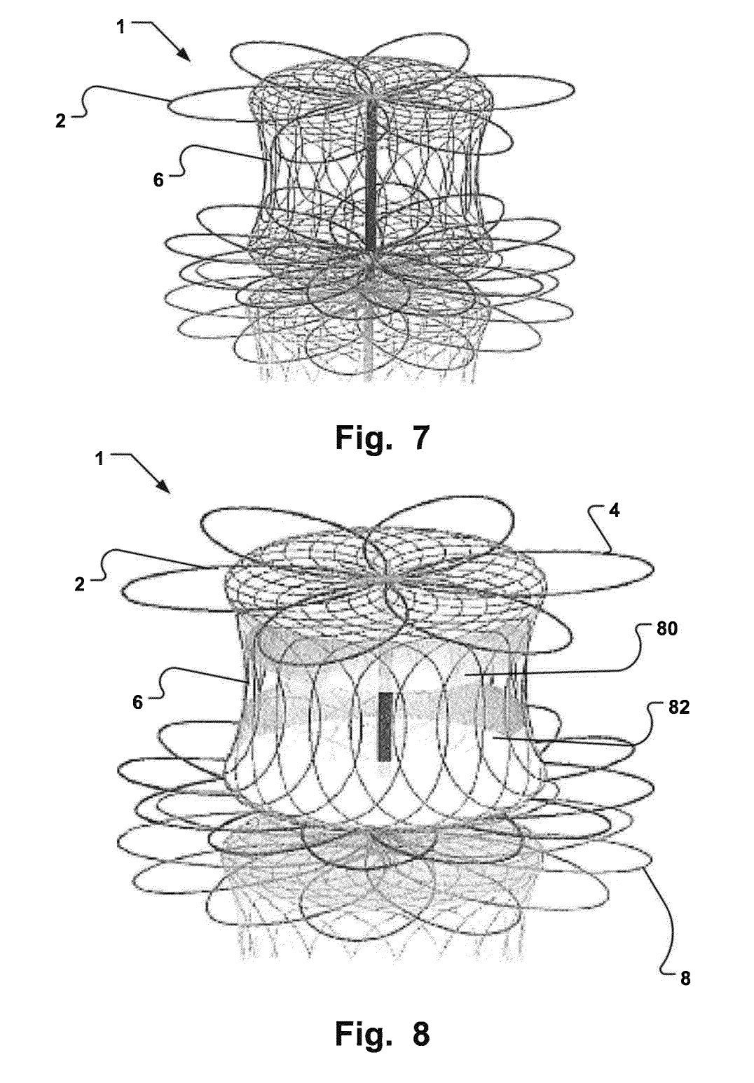

FIG. 7 shows a device 1 for occluding an opening in a body. In this Figure, the first element 2 has been joined with the second element 6.

In one example, an end of the first element 2 is joined with a corresponding end of the second element 6 by a technique, such as welding, pinching the ends together, or hooking the ends together.

In another example, also another end of the first element 2 is joined with a corresponding end of the second element 6 by a technique, such as welding, pinching the ends together, clamping the ends together or hooking the ends together.

In some examples the first element 2 is joined with the second element 6 in an axial centre or a hub of the first and second elements 2, 6. By joining the first and second elements 2, 6 only in an axial centre or hub, the second element 6 can retain its flexibility.

In further examples, at least one element 2, 6 comprises a coating, such as accell™. The use of accell™ or other similar coatings enhances the adhesion and proliferation of fibroblasts and endothelial cells onto surfaces. It may also overcome certain materials cytotoxic issues. The use of accell™ or similar coatings may further provide faster endothelization.

In FIG. 4, which is another view of the device 1 seen from the top at an angle, it can be seen what happens with the device 1, when radial pressure is applied from two directions, indicated with the arrows. When radial pressure is applied to the second element 6 of the device 1, the second element 6 will be deformed. Even if very little pressure is applied to the second element 6, it will be deformed, since the second element has been made very flexible, by the use of braided material, small diameter of the thread or wire, use of certain material or any combination of these methods. However, the first element 2 is not affected by the radial pressure applied, since the second element 6 is only attached to or joined with the first element 2 in an axial centre, at the ends of the second element 6 in this example.

From FIG. 4, membranes 40, 42 can be seen. In the example depicted in FIG. 4, the membranes are located on the outside of the first and third sections 4, 8 of the first element 2, i.e. one membrane 40 at the top of the device 1 and one membrane at the bottom 42 of the device. In an alternative example, the membranes 40, 42 could instead be located on the other side of the first and third sections 4, 8 of the first element 2, i.e. one membrane wedged between the first section 4 of the first element 2 and the second element 6 and the other membrane 42 wedged between the third section 8 of the first element 2 and the second element 6. Alternatively, only one of the membranes 40, 42 may be used. As another alternative, the membranes 40, 42 may be attached to the inside of the first and third sections 4, 8 of the first element 2, i.e. a membrane 40 may be attached to the first section 4 from below in FIG. 4 and a membrane 42 may be attached to the third section 8 from above in FIG. 4.

FIG. 8 shows another view of the device 1, in which membranes 80, 82 are located inside the second element 6. In this example one membrane 80 is located slightly closer to the first section 4 of the first element 2 than the other membrane 82. The membrane 80 can be located close to the end of the second element 6 that faces the first section 4 of the first element 2 or close to the middle of the second element 6. Likewise, the membrane 80 can be located close to the end of the second element 6 that faces the third section 8 of the first element 2 or close to the middle of the second element 6. The membrane 80 may also be located at a certain distance from the membrane 82. In some examples, only one of the membranes 80, 82 may be used.

The membranes 40, 42, 80, 82 of the device 1 for occluding an opening in a body are preferably formed of a thin, flexible material, such as a fabric which may be folded taut without being damaged. Elastic polymeric materials such as nylon, polyester, polypropylene, polytetrafluoroethylene and expanded polytetrafluoroethylene, as well as natural fabrics such as silk or wool, should meet the requirements of the membrane. In one example which has been found to work well, the membranes 40, 42, 80, 82 are formed of a woven polyester. The membranes 40, 42, 80, 82 can also be made, at least partly, by a biodegradable material. The membranes 40, 42, 80, 82 facilitate thrombosis, since they are made of a dense material. The membranes may also provide an improved endothelialization.

Referring again to FIG. 3, the first section 4 of the first element 2 comprises a plurality of loops 5 in a first geometrical plane, which loops 5 are arranged in a regular pattern, with each loop 5 extending from an axial centre of the first geometrical plane, and each loop 5 being adjacent to another loop 5 on a first side in the first geometrical plane and also adjacent to another loop 5 on a second side in the first geometrical plane, so that the plurality of loops 5 together form the shape of a corolla. Further, from FIG. 3 it can also be seen that in one example, the third section 8 of the first element 2 comprises a plurality of loops 5 in a second geometrical plane, which loops 5 are arranged in a regular pattern, with each loop 5 extending from an axial centre of the second geometrical plane, and each loop 5 being adjacent to another loop 5 on a first side in the second geometrical plane and also adjacent to another loop 5 on a second side in the second geometrical plane, so that the plurality of loops 5 together form the shape of a corolla. By forming the first and third sections 4, 8 of the first element this way, a symmetrical design of the first section 4 as well as the third section 8 is achieved. In one example, the first geometrical plane is parallel with the second geometrical plane.

The plurality of loops 5 of the first section 4 of the first element 2 are, in one example, overlapping in the first geometrical plane. In another example, the plurality of loops 5 of the third section 8 of the first element 2 are overlapping in the second geometrical plane. Alternatively, the loops 5 in both the first and the second geometrical planes are overlapping.

In some examples, the plurality of loops 5 of the first section 4 are fewer than the plurality of loops 5 of the third section 8.

In some examples, the diameter of the second element 6 is smaller than the diameter of the first section 4 of the first element 2. This is advantageous, since the first section 4, when used, is preferably on the side of a septum or body wall.

In some examples, the diameter of the second element 6 is smaller than the diameter of the third section 8 of the first element 2. This is advantageous, since the third section 8, when used, is preferably on the side of a septum or body wall.

Alternatively, both the diameter of the first section 4 and the diameter of the third section 8 are larger than the diameter of the second element 6.

In one example, the diameter of the first section 4 of the first element 2 is larger than a diameter of the third section 8 of the first element 2. This may be advantageous in some situations, when a rupture needs to be sealed at a target site, where the space on one side of the rupture is more limited than on the other side of the rupture.

In some examples, the plurality of loops 5 are formed from one single thread or wire 3, Alternatively, the plurality of loops 5 are formed from a plurality of threads or wires 3, whereafter the plurality of threads or wires 3 are joined by a technique, such as welding. Other techniques for joining the threads or wires 3, such as pinching the plurality of threads or wires together, clamping the plurality of threads or wires together, or hooking the plurality of threads or wires together, could be used. By first forming the loops 5 from a plurality of threads or wires 3 and then joining the plurality of threads or wires 3, the manufacturing is simplified.

One example of this disclosure is a method of manufacturing a device 1 for occluding an opening in a body. This method comprises forming a first section 4 of a first element 2, or part thereof by forming at least one loop 5 from a single thread or wire 3 in a first geometrical plane. The method further comprises forming a second section 7 of a first element 2, or part thereof by extending the single thread or wire 3 perpendicularly from the first geometrical plane to a second geometrical plane, the second geometrical plane preferably being parallel to the first geometrical plane. Another step of the method is to form a third section 8 of the first element 2, or part thereof by forming at least one loop 5 from the single thread or wire 3 in the second geometrical plane. An option of the method is to join different parts 20 by a technique, such as welding, pinching the plurality of threads or wires 3 together, clamping the plurality of threads or wires 3 together, or hooking the plurality of threads or wires 3 together, each part 20 comprising parts of the first, second and third sections 4, 7, 8 of the first element 2, if only parts of the first, second and third sections 4, 7, 8 were formed in the previous steps. In the method, forming of a first section 10 of a second element 6 by braiding at least one thread or wire 9 with a first braid pitch may also be performed. Also forming of a second section 11 of a second element 6 by braiding at least one thread or wire 9 with a second braid pitch, which braid pitch is smaller than the first braid pitch is performed. A third section 12 of a second element 6 is also formed by braiding at least one thread or wire 9 with the first braid pitch. The first and second elements 2, 6 are thereafter joined by a technique, such as welding, pinching the plurality of threads or wires together, clamping the plurality of threads or wires together, or hooking the plurality of threads or wires together.

Another example of this disclosure is a medical procedure for occluding an opening in a body. The catheters 30, 32 used in the medical procedure and the device 1 are shown in FIG. 11. The medical procedure comprises positioning of a device 1 inside a restraining catheter 30. The medical procedure also comprises positioning of a pushing catheter 32 inside the restraining catheter 30 adjacent to the device 1, further away from a target site than the device 1. Thereafter the restraining catheter 30, the pushing catheter 32 and the device 1 are inserted into the body. A distal end of the restraining catheter 30 is positioned at the target site and a device 1 is positioned inside the body opening. Thereafter the device 1 is pushed through the restraining catheter 30 with the pushing catheter 32 until the device 1 has been released, so that a first section 4 of a first element 2 of the device 1 is positioned on an inside of a rupture to be sealed. Another step of the medical procedure involves removing of the pushing catheter 32. Thereafter, the restraining catheter 30 is removed, so that the first section 4 of the first element 2 of the device 1 is positioned on the inside of the rupture to be sealed with the device 1 and a third section 8 of the first element 2 is positioned on an outside of the rupture, whereby a second element 6 of the device 1 is returned to its preset shape and the device 1 thereby is radially contracted so as to close the rupture. The restraining catheter 30 can thereafter be removed from the body.

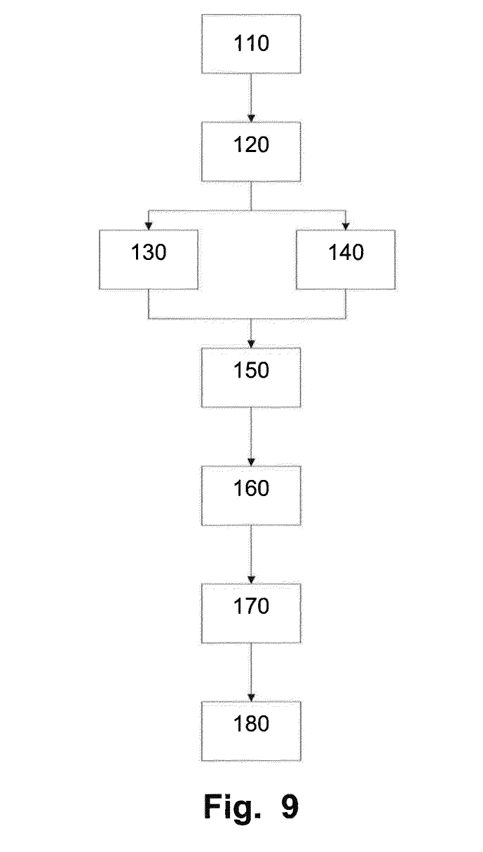

A further example of the disclosure is illustrated in FIG. 9. FIG. 9 shows the steps of a method of manufacturing a device 1 for occluding an opening in a body. In step 110, a first section 4 of a first element 2, or part thereof is formed by forming at least one loop 5 from a single thread or wire 3 in a first geometrical plane. In step 120 a second section 7 of a first element 2, or part thereof is formed by extending the single thread or wire 3 perpendicularly from the first geometrical plane to a second geometrical plane. The second geometrical plane is preferably parallel to the first geometrical plane. In step 130 a third section 8 of the first element 2, or part thereof is formed by forming at least one loop 5 from the single thread or wire 3 in the second geometrical plane. If only parts of the first and third sections 4, 8 were formed in steps 110 and 130, then the different parts 20 are joined by a technique, such as welding, pinching the plurality of threads or wires together, clamping the plurality of threads or wires together, or hooking the plurality of threads or wires together. This is shown in FIG. 9 with the step 140. In step 150, a first section 10 of a second element 6 is formed by braiding at least one thread or wire 9 with a first braid pitch. A second section 11 of a second element 6 is formed in step 160 by braiding at least one thread or wire 9 with a second braid pitch. The second braid pitch can be smaller than the first braid pitch. The second braid pitch could also be larger than the first braid pitch. In step 170 a third section 12 of a second element 6 is formed by braiding at least one thread or wire 9 with the first braid pitch. Thereafter, in step 180, the first and second elements 2, 6 are joined by a technique, such as welding, pinching the plurality of threads or wires together, clamping the plurality of threads or wires together, or hooking the plurality of threads or wires together. Instead or in addition to having different braid pitch for the different sections of the second element 6, the second element may have fewer threads, wires for the longitudinal section than for the end sections, i.e. the second section 11 of the second element 6 may comprise fewer threads or wires 9 than the first and the third sections 10, 12 of the second element 6.

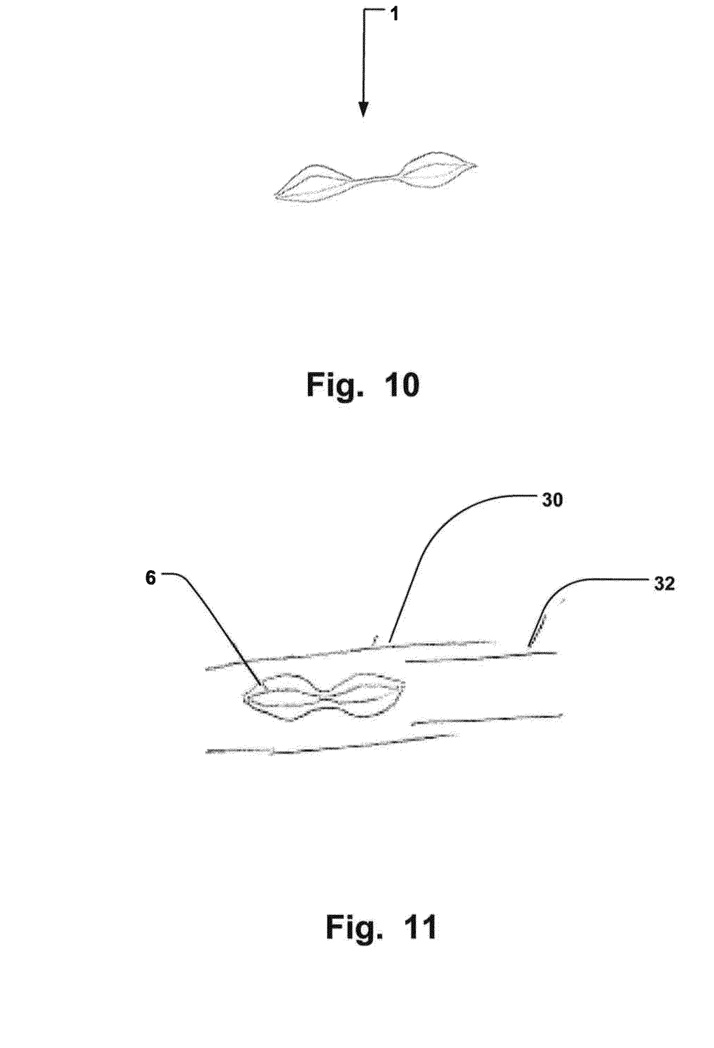

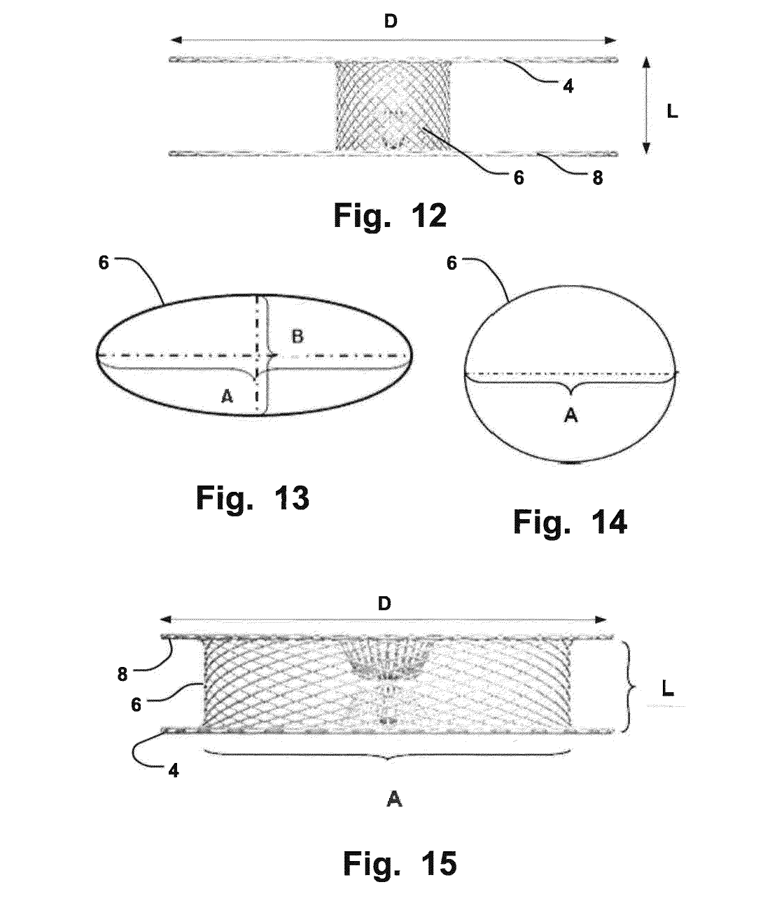

FIG. 10 shows the device 1 for occluding a body opening in its collapsed state. The device 1 is, preferably made of a shape memory material, such as nitinol. This is advantageous, since the device can then take on either a preset expanded configuration or a collapsed configuration. The device 1 can then be configured to be constrained to respective collapsed configuration for delivery to the target site and to at least partially return to the respective preset, expanded configuration at the target site when unconstrained. In some examples, the occluder is used for a postinfarction ventricular septal defect. This example is depicted in FIGS. 12 and 13. As can be seen from FIG. 13, the second element 6 has an oval-like shape or is shaped as an oval. In FIG. 13, the shorter diameter is denoted B, whereas the longer diameter is denoted A. As can be seen from FIG. 12, the distance between the first section 4 and the third section 8 of the first element 2, when the device is in its preset expanded configuration, is denoted L and the diameter of the first section 4 or the third section 8 of the first element 2 is denoted D. The ratio of diameter A to diameter B is preferably in the range of 1.8-5.15, whereas the ratio of the diameter D to the distance L is in the range of 1.4-5.6 and preferably in the range of 3.2-5.6. Furthermore, the ratio of D to A is preferably in the range of 1.3-2.2 and the ratio of D to B in the range of 2.3-6.3. An occluder having the above given ratios is particularly well suited for being used for a postinfarction ventricular septal defect. In this example, the distance between the first section 4 and the third section 8 of the first element 2 is preferably of a fixed size, but can be stretched according to the septum thickness.

In some examples, the occluder is used for a muscular ventricular septal defect. As can be seen from FIG. 14, the second element 6 has a circle-like shape or is shaped as a circle. In FIGS. 14 and 15, the diameter of the second element 6 is denoted A, whereas the diameter of the first section 4 or the third section 8 of the first element 2 is denoted D. The distance between the first section 4 and the third section 8 of the first element 2, when the device is in its preset expanded configuration, is denoted L. The ratio of A to D is preferably in the range of 0.14-2 whereas the ratio of the distance L to A preferably is in the range of 0.35-1.75. Furthermore, the ratio of D to L is in the range of 1.4-5.6 and preferably in the range of 1.4-4. An occluder having the above given ratios is particularly well suited for being used for a muscular ventricular septal defect. For the Muscular VSD, the distance between the first section 4 and the third section 8 of the first element 2 is preferably of a fixed size, but can be stretched. This distance can also be shorter, and may then eliminate residual shunts or channels.

The present disclosure has been described above with reference to specific examples. However, other examples than the above described are equally possible within the scope of the disclosure. Different method steps than those described above, may be provided within the scope of the disclosure. The different features and steps of the disclosure may be combined in other combinations than those described. The scope of the disclosure is only limited by the appended patent claims. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

1. A device (1) for occluding an opening in a body, comprising:
a first element (2), comprising at least one thread or wire (3), and
a second element (6), such as a shaft section, said second element (6) being at least in a longitudinal section substantially circular or oval and comprising a braided material, said braided material comprising at least one thread or wire (9), and wherein a diameter of said second element (6) is of substantially the same size or smaller than said opening for all longitudinal sections, when said device (1) is in a preset, expanded configuration.

2. The device of claim 1, wherein said first element (2) comprises a first section (4), such as a non-braided disc-formed section, said first section (4) comprising at least one loop (5) of said thread or wire (3) extending from a central portion of said first section (4) to a peripheral portion of said first section (4) and returning back to said central portion.

3. The device of claim 2, wherein said first element (2) further comprises a second section (7), such as a tubular section, said second section (7) being connected to said first section (4) and said second section (7) having a smaller diameter than a diameter of said first section (4).

4. The device of claim 3, wherein said first element (2) further comprises a third section (8) such as a non-braided disc-shaped section, said third section (8) being connected to said second section (7) and said third section (8) comprising at least one loop (5).

5. The device of claim 4, wherein said at least one loop (5) of said first section (4) is shaped as a petal and wherein said at least one loop (5) of said third section (8) is shaped as a petal.

6. The device of claim 5, wherein said at least one thread or wire (3) is used for said first, second and third sections (4, 7, 8) of said first element, so that said at least one thread or wire (3) runs through said first, second and third sections (4, 7, 8).

7. The device of any of claims 5-6, wherein said first, second and third sections (4, 7, 8) of said first element are non-braided sections.

8. The device of any of claims 2-7, wherein said first element (2) is assembled with said second element (6) into one unit, said assembled unit comprising said first and second elements (2, 6).

9. The device of claim 8, wherein said first element (2) is assembled with said second element (6) through interweaving of said first element (2) with said second element (6), thereby allowing for a sliding movement of said first element (2) relative said second element (6).

10. The device of claim 8 or 9, wherein one end of said first element (2) is joined with a corresponding end of said second element (6) by a technique, such as welding, pinching said ends together, or hooking said ends together.

11. The device of claim 10, wherein another end of said first element (2) is joined with a corresponding end of said second element (6) by a technique, such as welding, pinching said ends together, clamping said ends together or hooking said ends together.

12. The device of claim 10 or 11, wherein said first element (2) is joined with said second element (6) in an axial centre or a hub of said first and second elements (2, 6).

13. The device of any of claims 1-11, wherein said braided material is braided so that said at least one thread or wire (9) of said braided material has a certain first braid pitch in a first section (10) of said second element (6) and a certain second braid pitch, different from said first braid pitch, in a second section (11) of said second element (6).

14. The device of claim 13, wherein said first braid pitch is larger than said second braid pitch.

15. The device of any of claims 1-14, wherein at least one element (2, 6) comprises a coating for enhancing the adhesion and proliferation of fibroblasts and endothelial cells onto surfaces.

16. The device of any of claims 6-15, wherein said first section (4) of said first element (2) comprises a plurality of loops (5) in a first geometrical plane, which loops (5) are arranged in a regular pattern, with each loop (5) extending from an axial centre of said first geometrical plane, and each loop (5) being adjacent to another loop (5) on a first side in said first geometrical plane and also adjacent to another loop (5) on a second side in said first geometrical plane, so that said plurality of loops (5) together form the shape of a corolla and wherein said third section (8) of said first element (2) comprises a plurality of loops (5) in a second geometrical plane, which loops (5) are arranged in a regular pattern, with each loop (5) extending from an axial centre of said second geometrical plane, and each loop (5) being adjacent to another loop (5) on a first side in said second geometrical plane and also adjacent to another loop (5) on a second side in said second geometrical plane, so that said plurality of loops (5) together form the shape of a corolla.

17. The device of claim 16, wherein said plurality of loops of said first section (4) of said first element (2) are overlapping in said first geometrical plane, and wherein said plurality of loops of said third section (8) of said first element (2) are overlapping in said second geometrical plane.

18. The device of any of claims 16-17, wherein said plurality of loops (5) of said first section (4) are fewer than said plurality of loops (5) of said third section (8).

19. The device of any of claims 6-18, wherein a diameter of said second element (6) is smaller than a diameter of said first section (4) of said first element (2).

20. The device of any of claims 6-19, wherein a diameter of said second element (6) is smaller than a diameter of said third section (8) of said first element (2).

21. The device of any of claims 6-20, wherein a diameter of said first section (4) of said first element (2) is larger than a diameter of said third section (8) of said first element (2).

22. The device of any of claims 16-21, wherein said plurality of loops (5) are formed from one single thread or wire (3) or wherein said plurality of loops (5) are formed from a plurality of threads or wires (3), and wherein said plurality of threads or wires (3) are joined by a technique, such as welding, pinching said plurality of threads or wires together, clamping said plurality of threads or wires together, or hooking said plurality of threads or wires together.

23. The device of any of claims 16-22, wherein said plurality of loops (5) are configured to at least partially radially surround said opening, when said device is in said preset, expanded configuration.

24. The device of any of claims 4-23, wherein said second element (6) has an oval shape with a shorter diameter (B), and a longer diameter (A) and wherein a ratio of said longer diameter (A) to said shorter diameter (B) is in the range of 1.8-5.15.

25. The device of any of claims 4-23, wherein said device has a diameter (D) of said first section (4) or said third section (8) of said first element (2) and wherein said device has a distance (L) between said first section (4) and said third section (8) of said first element (2), when said device is in its preset expanded configuration and wherein a ratio of said diameter (D) to said distance (L) is in the range of 1.4-5.6 and preferably in the range of 3.2-5.6.

26. The device of any of claims 4-23, wherein said device has a diameter (D) of said first section (4) or said third section (8) of said first element (2) and wherein said second element (6) has a diameter (A) and wherein a ratio of said diameters (D/A) is in the range of 1.3-2.2.

27. The device of any of claims 4-23, wherein said second element (6) has an oval shape with a shorter diameter (B), and a longer diameter (A) and wherein said device has a diameter (D) of said first section (4) or said third section (8) of said first element (2) and wherein a ratio of said diameter to said shorter diameter (D/B) is in the range of 2.3-6.3.

28. The device of any of claims 4-23, wherein a distance between said first section (4) and said third section (8) of said first element (2) is preferably of a fixed size, but is stretchable according to the septum thickness.

29. The device of any of claims 4-23, wherein said device has a diameter (D) of said first section (4) or said third section (8) of said first element (2) and wherein said second element (6) has a diameter (A) and wherein a ratio of said diameters (A/D) is in the range of 0.14-2.

30. The device of any of claims 4-23, wherein said device has a distance (L) between said first section (4) and said third section (8) of said first element (2), when said device is in its preset expanded configuration and wherein said second element (6) has a diameter (A) and wherein a ratio of said distance (L) to said diameter (A) preferably is in the range of 0.35-1.75.

31. The device of any of claims 4-23, wherein said device has a diameter (D) of said first section (4) or said third section (8) of said first element (2) and wherein said device has a distance (L) between said first section (4) and said third section (8) of said first element (2), when the device is in its preset expanded configuration and wherein a ratio of said diameter (D) to said distance (L) is in the range of 1.4-5.6 and preferably in the range of 1.4-4.

32. The device of any of claims 4-23, wherein a distance between the first section (4) and said third section (8) of said first element (2) is of a fixed size, and stretchable.

33. The device of any of claims 4-23, wherein said first section (4) and said third section (8) are disposed non-coaxially with respect to one another in said preset, expanded configuration.

34. The device of any of claims 4-23, wherein one of said first section (4) and said third section (8) of said first element (2) is configured to cover an opening of a ventricular septal defect in the right atrium, and wherein the other of said first section (4) and said third section (8) of said first element (2) is configured to cover a corresponding opening of said ventricular septal defect in the left atrium.

35. The device of any of claims 4-34, wherein at least one of said first and second elements (2, 6) comprises a shape memory material.

36. The device of any of claims 4-35, wherein said device is provided with an adapter (14) for connecting said device (1) to a wire for delivery and/or retrieval.

37. The device of any of claims 4-36, wherein a material of said first element (2) is stiffer than a material of said second element (6).

38. The device of any of claims 4-37, wherein said plurality of loops (5) are configured to at least partially radially surround said opening, when said device is in said preset, expanded configuration.

39. The device of any of claims 4-38, wherein said thread or wire (3, 9) of said first element (2) and/or second element (6) has a diameter that varies along its length.

40. The device of any of claims 4-39, wherein said thread or wire (3, 9) of said first element (2) and second element (6) each comprises a shape memory material having an initial, expanded configuration corresponding to a preset, expanded configuration, and wherein said thread or wire (3, 9) of said first element (2) and second element (6) are configured to be constrained from their preset, expanded configurations to their reduced configurations for delivery to the target site and to self-expand and at least partially return to their respective preset, expanded configurations at a target site when unconstrained.

41. The device of any of claims 4-40, wherein a longitudinal section of said second element (6) has a non-concentric oval or elliptic shape in a cross-section perpendicular to a centre axis of the longitudinal section.

42. The device of any of claims 4-41, wherein said first element (2) of said device is formed substantially as a cone with the centre of the cone extending slightly inwardly towards the centre of the device.

43. A method of manufacturing a device (1) for occluding an opening in a body, comprising:
forming a first section (4) of a first element (2), or part thereof;
forming a second section (7) of a first element (2), or part thereof;
forming a third section (8) of a first element (2), or part thereof;
optionally joining different parts (20); and
forming first, second and third sections (10, 11, 12) of a second element (6) by braiding so that a diameter of said second element (6) is of substantially the same size or smaller than said opening for all longitudinal sections, when said device (1) is in a preset, expanded configuration; and
joining said first and second elements (2, 6).

44. The method of manufacturing a device (1) for occluding an opening in a body according to claim 43, wherein said forming a first section (4) of a first element (2), or part thereof is performed by forming at least one loop (5) from a single thread or wire (3) in a first geometrical plane.

45. The method of manufacturing a device (1) for occluding an opening in a body according to claim 44, wherein said forming a second section (7) of a first element (2), or part thereof is performed by extending said single thread or wire (3) perpendicularly from said first geometrical plane to a second geometrical plane, said second geometrical plane preferably being parallel to said first geometrical plane.

46. The method of manufacturing a device (1) for occluding an opening in a body according to claim 45, wherein said forming a third section (8) of a first element (2), or part thereof is performed by forming at least one loop (5) from said single thread or wire (3) in said second geometrical plane.

47. The method of manufacturing a device (1) for occluding an opening in a body according to claim 46, wherein said optionally joining different parts (20) is performed by a technique, such as welding, pinching said plurality of threads or wires together, clamping said plurality of threads or wires together, or hooking said plurality of threads or wires together, each part (20) comprising parts of said first, second and third sections (4, 7, 8) of a first element (2).

48. The method of manufacturing a device (1) for occluding an opening in a body according to claim 47, wherein said forming a first section (10) of a second element (6) is performed by braiding at least one thread or wire (9) with a first braid pitch.

49. The method of manufacturing a device (1) for occluding an opening in a body according to claim 48, wherein said forming a second section (11) of a second element (6) is performed by braiding at least one thread or wire (9) with a second braid pitch, said second braid pitch being smaller than said first braid pitch.

50. The method of manufacturing a device (1) for occluding an opening in a body according to claim 49, wherein said forming a third section (12) of a second element (6) is performed by braiding at least one thread or wire (9) with said first braid pitch.

51. The method of manufacturing a device (1) for occluding an opening in a body according to claim 50, wherein said joining said first and second elements (2, 6) is performed by a technique, such as welding, pinching said plurality of threads or wires together, clamping said plurality of threads or wires together, or hooking said plurality of threads or wires together.

52. Use of the device of any of claims 1-42, for occluding a ventricular septal defect, said ventricular defect for instance being a muscular ventricular septal defect, a postinfarction ventricular septal defect, a myocardial infarction complication, a rupture of the intraventricular septum, a shunt from the left side of the heart to the right side of the heart, a perimembranous ventricular septal defects, e.g. located in the left ventricle outflow tract beneath the aortic valve, or a congenital cardiac anomaly.

53. A medical procedure for occluding an opening in a body, comprising:
positioning a device (1) inside a restraining catheter (30);
positioning a pushing catheter (32) inside said restraining catheter (30) adjacent to said device (1), further away from a target site than said device (1);
inserting said restraining catheter (30), said pushing catheter (32) and said device (1) into said body;
positioning a distal end of said restraining catheter (30) at said target site and a device (1) inside said body opening;
pushing said device (1) through said restraining catheter (30) with said pushing catheter (32) until said device (1) has been released, so that a first section (4) of a first element (2) of said device (1) is positioned on an inside of a rupture to be sealed;
removing said pushing catheter (32);
removing said restraining catheter (30), so that said first section (4) of said first element (2) of said device (1) is positioned on said inside of said rupture to be sealed with said device (1) and a third section (8) of said first element (2) is positioned on an outside of said rupture, whereby a second element (6) of said device (1) is returned to its preset shape and said device (1) thereby is radially contracted so as to close said rupture; and
removing said restraining catheter (30) from the body.
